(19) United States
(12) Patent Application Publication
(10) Pub. No.: US 2025/0262168 A1
(43) Pub. Date: Aug. 21, 2025

(54) COMBINATION THERAPEUTIC REGIMENS WITH 1,6-DIBROMO-1,6-DIDEOXY-DULCITOL

(71) Applicant: Eleison Pharmaceuticals LLC, Princeton, NJ (US)

(72) Inventors: Patrick Maguire, Foster City, CA (US); Robert Johnston, Princeton, NJ (US); Seth Lederman, New York, NY (US)

(73) Assignee: Eleison Pharmaceuticals LLC, Princeton, NJ (US)

(21) Appl. No.: 19/066,610

(22) Filed: Feb. 28, 2025

Related U.S. Application Data
(63) Continuation of application No. 17/606,026, filed on Oct. 23, 2021, now abandoned.
(60) Provisional application No. 62/837,761, filed on Apr. 24, 2019.

(51) Int. Cl. A61K 31/047; A61K 31/196; A61K 31/4045; A61K 31/4184; A61K 31/495; A61K 31/506; A61K 31/69; A61P 35/00

(52) U.S. Cl. CPC A61K 31/047; A61K 31/196; A61K 31/4045; A61K 31/4184; A61K 31/495; A61K 31/506; A61K 31/69; A61P 35/00

(57) ABSTRACT

Methods of treating a subject suffering from a brain tumor are described herein wherein said methods comprise administering a therapeutically effective amount of a crystalline polymorph of 1,6-dibromo-1,6-dideoxy-dulcitol (DBD) including combination therapies with synergistic second cancer treatments. Also disclosed are methods of screening subjects sensitive to a crystalline DBD polymorph and treating those subjects demonstrating sensitivity.

### CROSS-REFERENCE TO RELATED APPLICATIONS

This application is a Continuation Application of U.S. Ser. No. 17/606,026, filed Oct. 23, 2021, which is a § 371 National Stage Application of PCT/US20/29350 filed on Apr. 22, 2020, which claims priority to US 62/837,761 filed on Apr. 24, 2019, which are all hereby incorporated by reference in their entirety.

### FIELD OF THE INVENTION

Methods of treating cancer by administering 1,6-dibromo-1,6-dideoxy-dulcitol (dibromodulcitol or DBD), including crystalline DBD polymorphs, in combination with specific anti-cancer moieties is shown to have enhanced safety and efficacy.

### INTRODUCTION

Cancer is the second leading cause of death in the United States, exceeded only by heart disease. Despite recent advances in cancer diagnosis and treatment, surgery and radiotherapy may be curative if a cancer is found early, but current drug therapies for metastatic disease are mostly palliative and seldom offer a long-term cure. Even with new chemotherapies entering the market, the need continues for new drugs effective in monotherapy or in combination with existing agents as first line therapy, and as second and third line therapies in treatment of resistant tumors.

One example of a potential chemotherapeutic used to treat cancer is 1,6-dibromo-1,6-dideoxy-dulcitol (dibromo dulcitol or DBD). The crystal structure of DBD was first published by Simon and Sasvari in Acta. Cryst. (1971) B27, 806-815. Kellner et al., reported that DBD had a selective a vigorous antitumor effect. Kellner et al., “1,6-Dibromo-1,6-Dideoxy-Dulcitol: A New Antitumoral Agent,” Nature (1967) 28; 213 (5074):402-3. However, in these studies, DBD was prepared by treating dulcitol with aqueous hydrobromic acid saturated with gaseous hydrogen bromide at temperatures less than 00C. This process is no longer considered a safe method of making DBD. Moreover, it was reported in the literature that DBD was poorly soluble.

The present invention addresses the continued need to improve and develop new cancer treatments that have better safety and efficacy profiles.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject.

Described herein is the use of 1,6-dibromo-1,6-dideoxy-dulcitol, including crystalline DBD polymorphs, in combination with specific anti-cancer moieties to treat cancer. DBD has the molecular weight of 307.98 g/mol, the molecular formula C6H12Br2O4, and the following structure:

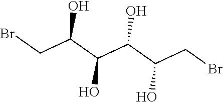 [SMILES: O[C@H]([C@H](O)[C@@H](O)CBr)[C@H](O)CBr]

Specifically, preferred embodiments include a method of treating a subject suffering from cancer tumor, wherein said method comprises administering a therapeutically effective amount of a crystalline polymorph of 1,6-dibromo-1,6-dideoxy-dulcitol (DBD). Examples of such cancer that can be treated as described herein include, but are not limited to adenocarcinoma, sarcoma, skin cancer, melanoma, bladder cancer, brain cancer, breast cancer, uterine cancer, ovarian cancer, prostate cancer, or lung cancer.

In preferred embodiments, the brain cancer is selected from an astrocytoma, meningioma, oligodendroglioma, mixed glioma and ependymoma. In further preferred embodiments, the brain tumor is a glioblastoma multiforme.

In further preferred embodiments, the subject is a human.

In even further preferred embodiments, the method further comprises administering a second cancer treatment selected from Temozolomide, radiation, ABT-888, Bortezomib, Imatinib, Panobinostat or BIBR-1532. In such embodiments, the DBD crystalline polymorph works synergistically with the second cancer treatment. In further embodiments, the method of claim 6, wherein the radiation therapy is delivered by a radiation-delivering system, including a gantry-based system, a robotic radiosurgery system, a subcutaneous implant, or a radioisotope.

In other preferred embodiments, the method further comprises: (A) obtaining or having obtained glioma cells from the subject; (b) testing or having tested the glioma cells in vitro for sensitivity to the DBD crystalline polymorph; and (c) administering the DBD crystalline polymorph to the subject who has demonstrated sensitivity in step (b). Performing this method may identify agents in combination with the DBD crystalline polymorph that have improved activity and enhanced efficacy at potentially lower doses leading to improved safety and fewer side effects.

### DESCRIPTION OF THE DRAWINGS

Embodiments are illustrated by way of example (and not limitation) in the figures of the accompanying drawings, in which like references, indicate similar elements and in which:

FIG. 1: Histogram from T98 cell cultures demonstrating the percentages of living cells at five and eight days following crystalline DBD polymorph treatment, compared to the non-treated cells considered 100% viability.

FIG. 2: Histogram from U373 cell cultures demonstrating the percentages of living cells at five and eight days following crystalline DBD polymorph treatment, compared to the non-treated cells considered 100% viability.

### DETAILED DESCRIPTION OF THE INVENTION

While certain embodiments of the present invention have been shown and described herein, it will be obvious to those skilled in the art that such embodiments are provided by way of example only. Numerous variations, changes, and substitutions will occur to those skilled in the art without departing from the invention. It should be understood that various alternatives to the embodiments described herein are, in some circumstances, employed in practicing the invention. It is intended that the following claims define the scope of the invention and that methods and structures within the scope of these claims and their equivalents be covered thereby.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

### DEFINITIONS

Unless defined otherwise, all technical and scientific terms used herein have the same meaning as commonly understood by those of ordinary skill in the art, such as in the arts of peptide chemistry, cell culture, chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4th ed., John Wiley & Sons, Inc.), which are incorporated herein by reference.

As used in this specification and the appended claims, the singular forms “a,” “an” and “the” include plural nouns unless the content clearly dictates otherwise. For example, reference to “a chemotherapeutic” includes a mixture of two or more such chemotherapeutics or a plurality of such chemotherapeutics.

As used herein, the term “comprise” or variations thereof such as “comprises” or “comprising” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein, the term “comprising” is inclusive and does not exclude additional, unrecited integers or method/process steps.

In embodiments of any of the compositions and methods provided herein, “comprising” may be replaced with “consisting essentially of” or “consisting of”. The phrase “consisting essentially of” is used herein to require the specified integer(s) or steps as well as those which do not materially affect the character or function of the claimed invention. As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

As used herein, “DBD” refers to 1,6-dibromo-1,6-dideoxy-dulcitol having the crystal structure as reported in the literature in Acta. Cryst. (1971) B27, 806-815.

The terms “crystalline DBD polymorph” or “crystalline polymorph” or “crystalline polymorphic form of DBD” refers to a crystalline form of 1,6-Dibromo-1,6-dideoxy-dulcitol as described in WO2016/205299 and US 2018/0362427, herein incorporated by reference in its entirety.

The term “subject”, as used herein in reference to individuals suffering from cancer and encompasses mammals and non-mammals. In a preferred embodiment, the subject is a human.

The terms “effective amount”, “therapeutically effective amount” or “pharmaceutically effective amount” as used herein, refer to an amount of at least one agent or compound being administered that is sufficient to treat cancer. The result is the reduction and/or alleviation of the signs, symptoms, or causes of such disease, or any other desired alteration of a biological system. For example, an “effective amount” for therapeutic uses is the amount of the composition comprising a compound as disclosed herein required to provide a clinically significant decrease in a disease. An appropriate “effective” amount in any individual case is determined using techniques such as a dose escalation study. Additionally, “effective amount”, “therapeutically effective amount” or “pharmaceutically effective amount” means that compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

In the present invention, a “tumor” or “cancer” is defined as a population of heterogeneous cells, collectively forming a mass of tissue in a subject resulting from the abnormal proliferation of malignant cancer cells. Thus, a “tumor” will contain both normal or “non-cancerous” cells and “cancer” or “cancerous” cells.

As used herein, “and/or” is to be taken as specific disclosure of each of the two specified features or components with or without the other. For example, “A and/or B” is to be taken as specific disclosure of each (i) A, (ii) B and (iii) A and B, just as if each is set out individually.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

### EXAMPLES

The invention will now be further illustrated with reference to the following examples. It will be appreciated that what follows is by way of example only and that modifications to detail may be made while still falling within the scope of the invention.

In the following Examples, the following abbreviations are used:

DEFINITIONS AND ABBREVIATIONS

| | |
|---|---|
| A2.5 | ABT-888 concentration 2.5 μM |
| A10 | ABT-888 concentration 10 μM |
| B1 | Bortezomib concentration 1 nM |
| B2.5 | Bortezomib concentration 2.5 nM |
| B5 | BIBR1532 concentration 5 μM |
| B10 | BIBR1532 concentration 10 μM |
| CTG | CellTiter-Glo (viability assay) |
| DBD | Dibromodulcitol |
| DMSO | DiMethyl SulfOxide (solvent for drugs, used as control) |
| GBM | Glioblastoma Multiforme |
| GBM(rec) | Recurrent Glioblastoma Multiforme |
| GSC's | Glioma serum-free Stem-like Cell cultures |
| IC50 | half maximal inhibitory concentration |
| I4 | Imatinib concentration 4 μM |
| I15 | Imatinib concentration 15 μM |
| MGMT | O6-methylguanine-methyltransferase |
| NT | Non-Treated control cell culture |
| P5 | Panobinostat concentration 5 nM |
| P20 | Panobinostat concentration 20 nM |
| RLU | Relative Luminescence Units |
| RTX | radiotherapy |
| TMZ | Temozolomide |
| T50 | TMZ concentration 50 μM |
| T100 | TMZ concentration 100 μM |
| ½IC50 | half the concentration of the IC50 |
| 3 Gy | three gray radiation |
| 6 Gy | six gray radiation |

### Overview

The crystalline DBD polymorph was studied an in vitro system using two human glioma cell lines with an attempt to identify DBD's ability to decrease cell viability, and to identify synergy with other compounds. The following studies Examples 1-3 were performed. The following results are presented:
a. Demonstration of a suitable in vitro concentration range of the polymorphic DBD drug that can be used for other cell studies (Example 1);
b. Determination of IC 50 (half maximal inhibitory concentration) values on day 5, and day 8, over twenty patient derived cell free cultures (Example 2); and
c. Determination if the effect of the DBD (½ of the IC 50 dose and IC 50 dose) is enhanced in combination with TMX (temozolomide), radiation therapy, or other targeted therapies in a panel of twenty primary serum-free cell cultures (Example 3).

### Example 1: Determination of the Suitable Concentration Range of Crystalline DBD Polymorph DBD Using Two Glioma Cell Lines

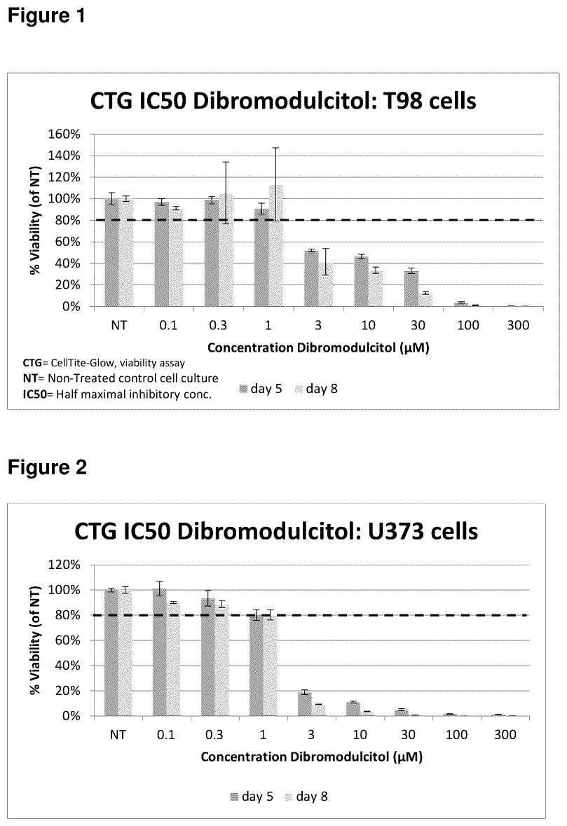

Since the concentration range of crystalline DBD polymorph to demonstrate effect in patient-derived serum-free glioma cell cultures is not yet known, a large range of concentrations was tested on two glioma cell lines. These lines were seeded in triplicate in a 96-wells plate, in a dilution of 500 cells/well. The ATP-based Cell Titer-Glo® (CTG) assays were performed at 5 and 8 days, to monitor cell growth and efficiency of DBD to effect decreased cell viability. FIGS. 1 and 2 show the histogram results of the two used glioma cell lines, T98 and U373.

Specifically, the histograms are normalized in percentages, where the non-treated cells have a 100% survival. The two lowest concentrations, 0.1 μM and 0.3 μM, were not effective. Moreover, almost all cells treated with 300 μM DBD dose are not viable. However, a concentration range between 1 and 100 μM was selected and was next tested on twenty patient-derived serum-free cell cultures.

### Example 2: Determination of the IC50 Values of Crystalline DBD Polymorph DBD in a Panel of Twenty Patient Derived Primary Serum-Free Cell Cultures

To assess the effects of crystalline DBD polymorph on the growth of patient derived GSC's (Glioma serum-free Stem-cell like cultures), dose-response assays were performed on twenty cultures.

For determining the IC50 of crystalline DBD polymorph, concentrations from 1 μM to 100 μM were applied. A dose-dependent decrease in viability was found, and the IC50 values of could be calculated using linear regression analysis. An overview of all IC50 values (μM) of DBD on the twenty cell cultures from both day 5 and day 8 analysis is shown in Table 1. Specifically, looking at Table 1, a high variation of different IC50 values is seen in this panel of patient derived GSC's. In addition, no correlation is seen between the MGMT promotor methylation status and the amount of the IC50 value. (MGMT methylation status can be a prognostic, and in some studies, has significantly improved the survival rate in patients with unresectable glioblastoma multiforme, who received concomitant radiation therapy and Temozolomide).

However, over time, between five and eight days, cells get more sensitive to the drug and lower IC50 values are observed. See, for example GS102peri, wherein IC50 at 5 days is 41.77 while the IC50 at 8 days has decreased to 11.94. This is surprising as known mechanisms of decreased responsiveness of a tumor cell to a particular chemotherapeutic include (1) decreased uptake of agents into or increased export out of the cell; (2) increased inactivation of agents in the cell; (3) enhanced repair of the DNA damage produced by the alkylating agents; and (4) the absence of cellular mechanisms that produce cytotoxicity in response to DNA damage. Thus, to observe an increase in sensitivity to cell killing of a cancer cell to treatment of a DBD polymorph in combination of one of the described chemotherapeutics is surprising.

TABLE 1
Table 1: An overview of the measured IC50 values of DBD
in μM for all twenty primary serum-free cell cultures.

| GS# | Passage | MGMT promotor methylation status | IC50 5 d | IC50 8 d |
|---|---|---|---|---|
| GS102peri | 18 | (core) Methylated (tumor & cells) | 41.77 | 11.94 |
| GS104peri | 22 | (core) Methylated (tumor & cells) | 3.48 | 0.57 |
| GS184 | 15 | Methylated (tumor & cells) | 18.6 | 1.96 |
| GS186core | 14 | Unmethylated Methylated (tumor & cells) | 4.56 | 1.94 |
| GS186peri | 14 | (core) Unmethylated Methylated (tumor & | 4.24 | 2.28 |
| | | cells) | | |
| GS203 | 20 | Methylated (tumor & cells) | 28.68 | 10.53 |
| GS224 | 17 | Methylated (tumor & cells) | 33.28 | 10.87 |
| GS245 | 13 | Unmethylated (tumor & cells) | 6.58 | 3.35 |
| GS249 | 18 | Methylated (tumor) | 0.63 | 0.24 |
| GS257 | 13 | Unmethylated (tumor & cells) | 11.86 | 10.22 |
| GS261 | 11 | Methylated (tumor) | 3.44 | 1.85 |
| GS279core (1) | 13 | Methylated (tumor & cells) | 3.6 | — |
| GS279core (2) | 16 | (core) Methylated (tumor & cells) | 7.64 | — |
| GS279peri | 13 | Unmethylated (tumor & cells) | 31.49 | 8.33 |
| GS281 | 13 | Unmethylated (tumor & cells) | 10.56 | 5.55 |
| GS289 | 13 | Methylated (tumor) & Unmethylated (cells) | 6.88 | 3.22 |
| GS304 | 14 | (core) Methylated (tumor & cells) | 105.62 | 12.19 |
| GS323peri | 17 | Methylated (cells) | 4.75 | — |
| GS324core | 16 | Methylated (tumor & cells) | 8.96 | 2.98 |
| GS359 | 14 | Unmethylated (tumor & cells) | 11.84 | 7.27 |
| GS365 | 14 | MGMT promotor methylation status | 14.63 | 5.69 |

‘Core’ means cells obtained from the tumor core.
‘Peri’ means cells from the invading margin, or ‘periphery’ of the patient’s tumor.

### Example 2: Determination of Whether the Effect of the DBD Polymorph (½ of IC50 Dose and IC50 Dose) is Enhanced in Combination With TMZ (Temozolomide, a Chemotherapeutic Alkylating Agent, Used for Treatment Against Gliomas), RTX (Radiation Therapy) or any Other Compound, in a Panel of Twenty Primary Serum-Free Cell Cultures

Crystalline DBD polymorph treatment was combined with chemotherapy, radiotherapy and targeted inhibitors to determine whether the crystalline DBD polymorph form could: enhance efficacy of the conventional therapies; enhance efficacy when combined with newer ‘targeted’ therapies; or would make (a subset of) cultures more sensitive to this treatment.

Therefore, cultures were treated with:
a. Crystalline DBD polymorph at IC50 and at ½IC50 doses (determined at day 5); and
b. Two different concentrations of either Temozolomide, radiotherapy, ABT-888 (PARP (Poly ADP-ribose polymerase) inhibitor), Bortezomib (proteasome inhibitor), Imatinib (Bcr-Abl, PDGF and c-KIT receptor tyrosine kinase inhibitor), Panobinostat (pan-HDAC (histone deacetylase inhibitor) or BIBR1532 (telomerase inhibitor).

Tables 2 (Day 5) and 3 (Day 8) show the enhancement factors (viability percentage of most effective monotherapy divided by viability percentage, in combination with temozolomide or radiation therapy) for all used twenty cell cultures for all treatment combinations. In some cases, effects of crystalline DBD polymorph treatment (decreased cell viability) were seen compared to monotherapy Temozolomide, radiation, ABT-888, Bortezomib, Imatinib, Panobinostat or BIBR1532.

The ‘heat map’ (heat map=representation of data in the form of a table in which data values are represented as different colors) with ‘darker grey colors’ represents larger integer values within the cells of the table, making an easier visualization of the data. The ‘darker’ the data cell, the more effective the combination of both therapies compared to the most effective monotherapy. As is shown in Tables 2 and 3, both Imatinib and Panobinostat overall, have the highest enhancement factors.

### Summary and Conclusions

Chemical moieties, including therapeutic agents, may be synthesized to yield different crystalline structures. In many cases, these differing ‘polymorphs’, by virtue of their crystalline structure, can also have differing physicochemical properties.

With changes in physicochemical properties, the polymorph can also have differing therapeutic profiles, that can alter safety and efficacy of the delivered compound when used therapeutically in humans. This may affect the polymorphs use with other adjuvant or combination drugs and therapies. This is especially important with combination therapies for oncology conditions.

The methods and results presented have identified a therapeutic in vitro dose range in patient derived glioma cell cultures, and have demonstrated that these effects are not influenced by MGMT status when treated with the crystalline DBD polymorph as described in WO2016/205299 and US 2018/0362427.

Moreover, with regard to other combination therapies, treatment with the DBD polymorph enhanced the cytotoxic effect when combined with another alkylating agent, temozolomide (by 50%) and with radiation treatment (by 30%).

When combined with newer targeted therapies (described below), the combination of the DBD polymorph and the newer targeted therapies has shown an enhanced effectiveness in in vitro patient derived cell studies. Specifically, a selection of targeted drugs was tested in combination with DBD in the panel of 20 patient derived cell cultures. This selection covered the most important pathways in the initiation of and continued growth of the malignancy human glioblastoma, such as:
a. bcr/abl, c-kit and pdgfR Tyrosine kinase
b. HDAC
c. Telomerase inhibition
d. Proteasome inhibition
e. DNA repair (PARP) inhibition

From these results (high enhancement factor, most cases) were achieved with HDAC inhibition (Panobinostat) and inhibition of Ras/MapK pathway, Src/Pax/Fak/Rac pathway, PI/PI3K/AKT/BCL-2 pathway and JAK/STAT pathway combined (downstream effects of Bcr-Abl pathway), using the tyrosine kinase inhibitor, Imatinib.

Example 3-Incorporation of Disclosure WO2016/205299 (U.S. Pat. No. 20,180,362327)

As described in paragraphs [007-010] in WO2016/205299 (U.S. Pat. No. 20,180,362327), the DBD polymorph has a molecular weight of 307.98 g/mol and the molecular formula C₆H₁₂Br₂O₀.

In one aspect described herein are crystalline polymorphs of 1,6-dibromo-1,6-dideoxy-dulcitol characterized by peaks at 19.59° (100.00) and 24.380° (79.52) and 31.260° (8.32) and 34.500° (25.56) and 34.810° (22.83) and 39.260° (23.63) at 2θ±0.1°. In further embodiments, such a crystalline polymorph is further characterized by at least two peaks selected from 19.59° (100.00) and 24.380° (79.52) and 31.260° (8.32) and 34.500° (25.56) and 34.810° (22.83) and 39.260° (23.63) at 2θ±0.1°. In further embodiments, such a crystalline polymorph is further characterized by at least three peaks selected from 19.59° (100.00) and 24.380° (79.52) and 31.260° (8.32) and 34.500° (25,56) and 34.810° (22.83) and 39.260 (23.63) at 2θ±0.1°. In further embodiments, such a crystalline polymorph is further characterized by at least four peaks selected from 19.59° (100.00) and 24.380° (79.52) and 31.260° (8.32) and 34.500° (25.56) and 34.810° (22.83) and 39.260° (23.63) at 2θ±0.1°. In further embodiments, such a crystalline polymorph is further characterized by at least five peaks selected from 19.59° (100.00) and 24.380° (79.52) and 31.260° (8.32) and 34.500° (25.56) and 34.810° (22.83) and 39.260° (23.63) at 2θ±0.1°.

In yet further embodiments, the crystalline polymorph exhibits an x-ray powder diffraction pattern substantially the same as the x-ray powder diffraction pattern shown in FIG. 1 in WO2016/205299 (U.S. Pat. No. 20,180,362327). In further embodiments, the crystalline polymorph exhibits an x-ray powder diffraction pattern substantially the same as the x-ray powder diffraction pattern shown in FIG. 2 in WO2016/205299 (U.S. Pat. No. 20,180,362327). In yet further embodiments, the crystalline polymorph exhibits an x-ray powder diffraction pattern substantially the same as the x-ray powder diffraction pattern described in Table 1 in WO2016/205299 (U.S. Pat. No. 20,180,362327). In yet further embodiments, the crystalline polymorph exhibits a beta angle of 96° as compared to the literature reported beta angle of 98°.

In a related aspect described herein are crystalline polymorphs of 1,6-dibromo-1,6-dideoxy-dulcitol, characterized by an endothermic point onset at about 184.4° C. and peak at approximately 191° C. as determined by differential scanning calorimetry. In a further embodiment, the crystalline polymorph is characterized by a differential scanning calorimetry pattern substantially the same as the differential scanning calorimetry pattern shown in FIG. 3 and/or FIG. 4 in WO2016/205299 (U.S. Pat. No. 20,180,362327).

### REFERENCES

1. Chang, A. Y., et al., Induction chemotherapy of dibromodulcitol, Adriamycin, vincristine, tamoxifen, and Halotestin with methotrexate in metastatic breast cancer: an Eastern Cooperative Oncology Group Study (E1181). Am J Clin Oncol, 1998. 21 (1): p. 99-104.
2. Csetenyi, J., et al., The distribution of [3H]-dibromodulcitol in the central nervous system of patients with brain tumour. Eur J Cancer Clin Oncol, 1983. 19(10): p. 1389-92.
3. Berghauser Pont, L. M., et al., The Bcl-2 inhibitor Obatoclax overcomes resistance to histone deacetylase inhibitors SAHA and LBH589 as radiosensitizers in patient-derived glioblastoma stem-like cells. Genes Cancer, 2014. 5(11-12): p. 445-59.
4. Lee, J., et al., Tumor stem cells derived from glioblastomas cultured in bFGF and EGF more closely mirror the phenotype and genotype of primary tumors than do serum-cultured cell lines. Cancer Cell, 2006. 9(5): p. 391-403.
5. Fael Al-Mayhani, T. M., et al., An efficient method for derivation and propagation of glioblastoma cell lines that conserves the molecular profile of their original tumours. J Neurosci Methods, 2009. 176(2): p. 192-9.
6. Nikanjam, M., S. Liu, and R. Kurzrock, Dosing targeted and cytotoxic two-drug combinations: Lessons learned from analysis of 24,326 patients reported 2010 through 2013. Int J Cancer, 2016. 139(9): p. 2135-41.

1. A method of treating a subject suffering from cancer, wherein said method comprises administering a therapeutically effective amount of a crystalline polymorph of 1,6-dibromo-1,6-dideoxy-dulcitol (DBD) and a cancer treatment selected from Temozolomide, radiation, ABT-888, Bortezomib, Imatinib, Panobinostat or BIBR-1532 wherein the DBD polymorph works synergistically with the cancer treatment.

2. The method of claim 1, wherein the cancer is selected from adenocarcinoma, sarcoma, skin cancer, melanoma, bladder cancer, brain cancer, breast cancer, uterine cancer, ovarian cancer, prostate cancer, or lung cancer.

3. The method of claim 2, wherein said brain cancer is selected from an astrocytoma, meningioma, oligodendroglioma, mixed glioma and ependymoma.

4. The method of claim of 3, wherein the astrocytoma is a glioblastoma multiforme.

5. The method of claim 1, wherein said subject is a human.

6. (canceled)

7. The method of claim 1, wherein the radiation therapy is delivered by a radiation-delivering system, including a gantry-based system, a robotic radiosurgery system, a subcutaneous implant, or a radioisotope.

8. (canceled)

9. The method of claim 1 wherein said method further comprises:
a. Obtaining or having obtained glioma cells from the subject;
b. Testing or having tested the glioma cells in vitro for sensitivity to the DBD crystalline polymorph; and
c. Administering the DBD crystalline polymorph to the subject who has demonstrated sensitivity in step (b).

10. A composition used to treat a patient with cancer comprising administering a therapeutically effective amount the DBD crystalline polymorph.
